(19) United States
(12) Patent Application Publication
(10) Pub. No.: US 2005/0244865 A1
(43) Pub. Date: Nov. 3, 2005

(54) Molecular lithography with DNA nanostructures

(76) Inventors: Chengde Mao, West Lafayette, IN (US); Zhaoxiang Deng, West Lafayette, IN (US)

Correspondence Address: BAKER & DANIELS LLP, 300 NORTH MERIDIAN STREET, SUITE 2700, INDIANAPOLIS, IN, 46204, US

(21) Appl. No.: 11/085,998

(22) Filed: Mar. 22, 2005

Related U.S. Application Data
(60) Provisional application No. 60/555,733, filed on Mar. 23, 2004.

(51) Int. Cl. C12Q 1/68; C12M 1/34

(52) U.S. Cl. 435/6; 435/287.2; 427/2.11

(57) ABSTRACT

A method of making molecular lithographs and molecular lithographs prepared by a bottom-up synthesis of nanopatterns having discrimination at the level of several nanometers. Molecules such as DNA may be configured to pattern useful configurations. Lithographs enabling the reproduction of the patterns may be prepared according to the claimed method. Chemically bound molecules provide a mask for electrical connections that may be prepared by subsequent known processes from the lithograph.

Molecular patterns may be prepared by methods including bottom-up nanofabrication using DNA molecules. DNA molecules comprise sequences of the deoxyribonucleic acid and the nitrogenous bases adenine, guanine, cytosine and thymine. DNA of biological origin creates patterns for the molecules forming a living plant or creature. DNA may also be prepared synthetically sized to form a sequence of nitrogenous bases and molecular patterns

### DESCRIPTION OF THE FIGURES

FIG. 1 illustrates a pattern formed of molecules on a substrate.

FIG. 2 illustrates partially evaporated metal on the pattern molecules.

FIG. 3 illustrates a deposited-material, foil-lithograph formed on patterned molecules.

FIG. 4 illustrates the negative image of patterned molecules on a deposited-foil lithograph.

### DESCRIPTION OF THE INVENTION

The preparation of lithographs of molecules of the invention starts with depositing molecules on a first substrate. At a laboratory scale, a clean first substrate may be obtained by using freshly cleaved mica. As the instant molecular lithograph is of structures on the order of nanometers, airborne dust and debris may be orders of magnitude larger in size than the sought for molecular pattern. Accordingly, a clean first substrate facilitates the objective that the topography of the generated lithograph represents the sought for pattern and not interfering debris. Other suitable first substrates are possible provided the pattern molecules adsorb to the surface thereof, including glass, quartz, polymers, silicon. Silicon substrates have the advantage that the preparation of clean silicon substrates is well known and practiced on a commercial scale in the manufacture of semi-conductor chips. Moreover, the instant invention lends itself to the manufacture of semi-conductor chips as the configuration of conductors on the order of nanometers may be assembled from the bottom-up lithographs disclosed.

Deposit of the molecular pattern on the first substrate is conveniently accomplished by depositing a small volume of liquid containing the molecular pattern on the first substrate to permit the molecules to adsorb on the surface of the first substrate. Adsorption may occur in a few seconds to a minute or more. Adsorption can be expected at times from 20 seconds, possibly 10 seconds or 5 seconds, or if the attraction of the pattern molecules to the first substrate surface is sufficient, the adsorption may appear nearly instantaneous or in a sufficiently short time as to not be practically measurable. Adsorption may require a minute or more, possibly 2 minutes, or as long as 5 minutes before adequate adsorption of the pattern molecules occurs on the surface of the first substrate. Shorter adsorption times will be favored as providing less opportunity for contamination of the first substrate.

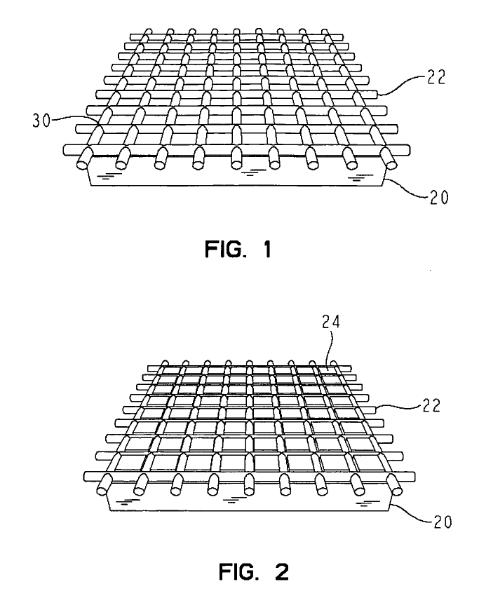

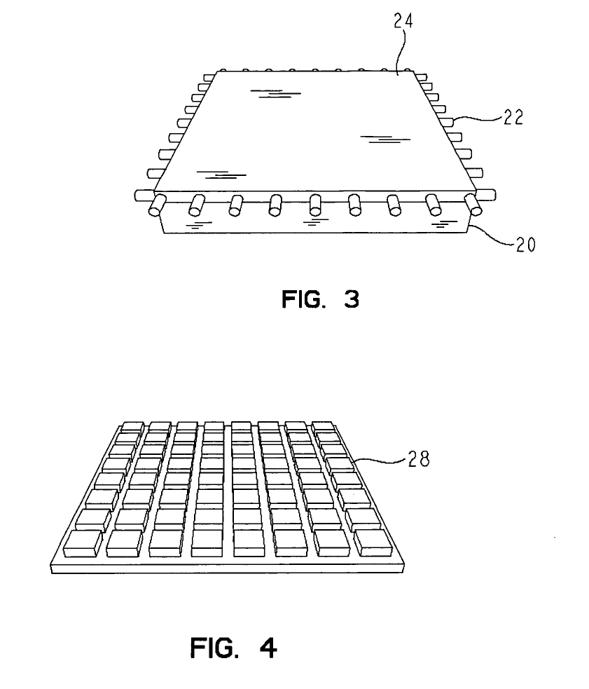

The invention is further described with the benefit to the FIGS. 1-4. The liquid containing the molecular pattern is advantageously washed from the surface of the first substrate 20 leaving the pattern molecules 22 adsorbed on the surface of the first substrate. The washing liquid is normally 10 mM Mg(CH₃CO₂)₂, which removes a large part of the buffer without adversely affecting the DNA structures.

The washing fluid may be dried from the molecular pattern and first substrate in ambient air. Drying may be accelerated by the use of compressed air, warmed air, or both.

The dried-first substrate with the pattern molecules adsorbed thereon on are next exposed to deposit of a material thereon. One deposit method is metal evaporation. Solid metal passes from a solid to a vapor state under the influence of sufficient heat in a vacuum. The pattern molecules and supporting first substrate comprise a cool condensing surface compared to the are exposed to vaporized material. The vaporized metal deposits on the pattern molecules and first substrate forming a film thereon 24. Gold is a suitable material for evaporation. Alternative deposit methods include chemical vapor deposition, plasma deposition, and electron-beam evaporation. Other useful deposition materials include silicon, copper, palladium, carbon, silver, nickel, and germanium.

The material deposition process may require from 30 minutes to as long as two hours to complete. The material vapor deposition itself requires from 1 to 5, most often near 2 minutes to complete a sufficient layer to form a lithograph. Time required beyond actual vapor deposition time is required to create an release the vacuum in the deposition instrument and instrument set-up.

Following creation of the film lithograph of the pattern molecules, an adhesive is applied to the film and a second substrate applied to the adhesive to form a multi-layer structure comprising the first substrate with the pattern molecules thereon, the film lithograph, the adhesive, and the second substrate. If the adhesive is of a setting type, then it is permitted to solidify before removing the lithograph 28, adhesive and second substrate are peeled from the first substrate. Alternatively, a non-setting type adhesive such as a pressure-sensitive adhesive may be employed to bind the second substrate to the lithograph, e. g., double sided-adhesive tape. Further, if the adhesive is of a setting-type, the adhesive itself may also serve the role of the second substrate. Such setting-type adhesive that may serve as a second substrate could be a thermoset resin such as an epoxy resin, or a polyester resin, or a vinyl ester resin.

Suitable pattern forming molecules are any molecules that may be designed for bottom-up nanofabrication. Such molecules may include dendrimers, carbon nanotubes and DNA of a biologic source, or synthetic DNA. Synthetic DNA is commercially available from enterprises such as Integrated DNA Technologies, Inc., Coralville, Iowa 52241 USA. DNA can be prepared to a customer's specification including areas of overlap 30 such as would generate an electrical connection if the lithograph were used as a template to prepare an integrated circuit. Such specifications may include the distance between overlap, the number of overlapping connections, and the number of connections to a junction.

### EXAMPLE 1

DNA nanostructures were assembled from single DNA strands. The synthesized single strands are purified by denaturing polyacrylamide gel electrophoresis. Denatured DNA is known. Denaturing is accomplished by immersion of the DNA strands in 40% by wt urea followed by raising the temperature to 55° C.

Purified DNA strands were combined in equimolar amounts and annealed by cooling from 95° C. to 22° C. for 48 hours to form molecular structures of anti-parallel, double-cross-over (DAEE) DNA.

A sample of 2 μl of the annealed DNA was spotted on freshly cleaved mica substrate and permitted to adsorb for 10 seconds. After adsorption, the mica and adsorbed DNA is washed with 30 μl of 10 mM Mg(CH₃CO₂)₂.

### EXAMPLE 2

After drying, the adsorbed DNA and mica substrate of Example 1 were examined by atomic-force microscopy (AFM) in tapping mode at a tip speed of 10 μm/s using a Nanoscope IIIa controller available from Veeco Instruments Inc. Digital Instruments 100 Sunnyside Blvd. Ste. B Woodbury New York, 11797-2902, USA, and a 10-nm probe. Examination of the AFM images confirmed the formation of a molecular pattern. The DAEE arrays were 12 nm in width measured as full width of the AFN plot at one-half the maximum plot height. The height of the DAEE array was determined by section analysis to be 0.8 nm.

### EXAMPLE 3

The sample prepared according to Example 1 was placed in a thermal evaporator (Turbo Vacuum Evaporator-EFFA, Ernest F. Fullam, Inc., 900 Albany Shaker Rd. Latham, N.Y. 12110-1491, USA) using gold as the deposited material. The deposit speed was adjusted to deposit gold at the rate of 0.2 nm/s. The deposition continued for about 2 minutes until a gold film of 20 nm thickness was deposited.

After the sample was removed from the thermal evaporator, a drop of pre-mixed epoxy-resin adhesive was placed on gold film of the sample. The epoxy resin was then covered with a glass slide. After the adhesive set, the glass slide with the attached adhesive and gold film lithograph was peeled from the mica substrate.

### EXAMPLE 4

The gold film lithograph of Example 3 was then examined with AFM. The depth of grooves formed by DAEE DNA was measured at 0.5 nm. The width of the DAEE DNA groove was measured as 4 nm, a dimension smaller that the tip width of the AFM. Apparent width measured as full width at half maximum height of the AFM plot was found to be 9 nm, which compared favorably with the measured value of the projection formed by the DAEE DNA on the mica substrate of 12 nm.

The lithograph accurately replicated the pattern formed by the pattern forming DAEE DNA molecules.

1. A lithograph comprising a molecular pattern.

2. The lithograph of claim 1 comprising a molecular pattern the lithograph pattern having a width dimension from 4 to 100 nm.

3. The lithograph of claim 1 wherein the molecular pattern is formed of molecules of DNA.

4. The lithograph of claim 3 wherein the pattern of DNA is engineered to form an electrical circuit.

5. A circuit formed from the lithograph of claim 4.

6. A lithograph of claim 1 comprising deposited material selected from the group consisting of silicon, copper, palladium, gold, carbon, silver, nickel and germanium.

7. The lithgraph of claim 2 wherein the lithgraph pattern is from 4 to 20 ηm.

8. A method of making a lithograph comprising
a.) depositing one or more molecular structures on a first substrate;
b) depositing a film on the substrate;
c) adhering an adhesive to the deposited material;
d) adhering a second substrate to the adhesive;
e) removing the deposited material, adhesive, and second substrate from the first substrate.

9. The method of claim 8 wherein the molecular structures comprises of at least one molecule of DNA.

10. The method of claim 8 deposited material is selected from the group comprising: gold, platinum, palladium, aluminum, iron, and alloys thereof.

11. The method of claim 8 wherein the adhesive is selected from the group consisting of polyurethane, epoxy, thermoset polyester resin, thermoset vinyl ester resin, cyanoacrylate resin,

12. The method of claim 8 wherein the second substrate is selected from the group consisting of glass, a polymeric sheet, metal, polymeric coated metal.

13. The method of claim 8 wherein the material deposited is deposited by plasma deposition, or thermal evaporation.

14. The method of claim 8 wherein the second substrate is a thermoset polymer and consists of the adhesive.

15. The method of claim 8 wherein the adhesive is a pressure-sensitive adhesive.
